(19) United States
(12) Patent Application Publication
(10) Pub. No.: US 2006/0068028 A1
(43) Pub. Date: Mar. 30, 2006

(54) Means of prevention for HIV/AIDS infections, grippe and flu

(76) Inventor: Yakov Brilman, Voorhees, NJ (US)

Correspondence Address: Yakov Brilman, 219 Laurel Rd apt. 508, Voorhees, NJ, 08043, US

(21) Appl. No.: 10/972,490

(22) Filed: Sep. 27, 2004

(51) Int. Cl. A61K 33/22

(52) U.S. Cl. 424/660

(57) ABSTRACT

For the prevention of HIV/AIDS and venereal diseases, grippe and flu, it is proposed to use the offered solutions and other creams possessing virucide and bactericidal characteristics for processing the possible infected area of the skin and mucous membranes of the body, and also solutions for inhalation for the grippe and flu.

In addition to existing means of the prophylaxy HIV/AIDS diseases, grippe and flu I am also offering using the disinfecting solution and cream. They conduct processing on places on the skin and mucous shell of the body where infection can enter on organism after contact with a sick person or virus carrier of these infections.

To achieve the disinfecting effect is the offered solution and cream containing active chlorine in safe concentration and which quickly kills the virus of these infections.

Pantocid (Pantosept) is suggested in combination with other necessary substances.

The known use of Pantocid (Pantosept) in tablet form is for disinfection in water, and for irrigation and cleaning of wounds (0.1-0.5% solution).

The solution Pantosept is the suggested use in a mixture with Sodium Tetraborate and hypertonic solution of the Sodium Chloride and Sodium Hydrocarbonate. The mixture hypertonic solution of the Sodium Chloride (4%) will promote the best cleansing from possible presence on mucous membranes and skin of the infected material (mucous, sperm and etc.) due to its osmatic effect. Besides, hypertonic salt solution is a germicidal characteristic.

Hydrocarbonate Sodium is also used for mouthwash, and to treat head colds, conjunctivitis, laringitis stomatitis, and etc.

Its presence in 5% solutions will promote best dilution Pantocid (Pantosept) in water, will increase the osmotic effect of the solution and promote the best cleansing of slime and other infected elements. Together with the hypertensive solution of Sodium Chlorine will increase the effect of rejecting the virus from cell tissue. Together with this the substance containing Chlorine (Pantocid) in solution with increased presence of alkaline is capable of bringing to a quick destruction of the virus. To increase the antiseptic effect of the solution add 0.5% Sodium Tetraborate, which is also known as germicidal preparation and put in practice for mouthwashes.

The suggested-solution to prevent HIV/AIDS infection is the following: 0.2% Pantocidi (Pantosept), 4% Sodium Chloride, 5% Hydrocarbonate Sodium and 0.5-1.0% Sodium Tetraborate on distilled water—apply externally of the preventive means solution “B” (Brilman's solution)

| | |
|---|---|
| Rp: Pantosept | 0.2 |
| Sodium Chloride | 4.0 |
| Sodium hydrocarbonate | 5.0 |
| Sodium tetraborate | 0.5 |
| Distilled Water | 100.0 |

MDS - External (for washing, mouthwashes, irrigation

The proposed composition of the solution “B”can be made in the form of powder or tablets to dissolve in above correlation in water.

This solution, for broad use, should be filled in available pharmacies with an adapted capacity for irrigation, wash, spray, etc. in the necessary volume.

With this solution is used a partial disinfection processing and also cleaning the skin and mucous membranes including the mouth, sexual organs, anus and crotch, and also any infected material by use of the mouth washes, washing and irrigation (washing vagina) after contact (sexual relations) with possible virus carrier HIV/AIDS diseases.

After washing with this solution I suggest to apply externally the unguentum (cream or gel) Pantosept 0.2%.

This medicine is put on the skin and mucous membranes of the sexual organ, anus and crotch for 15-20 min.

The earlier these proposed preventive means are used after sexual relations, the more chances there are for avoiding contamination.

Considering that in early stages of the contamination HIV/AIDS infection there occurs blocking of this virus in the bowels with mass destruction by the virus of “immune cells of the memory”—CD⁴+, it is necessary also with preventive and medical purpose using on tablets Pantocidi, possessing virulicidi characteristic. These tablets containing 40 mg. Pantocidi are offered to take once a day with water for 2 weeks or more with subsequent and periodic washing of the bowels 0.1-0.2% solution Pantocidi or sol. “B-2” (from the receipt of the solution “B” is excluded Sodium Tetraborate).

The medicine is to be taken externally using these facillities as indicated above after sexual relations, it can also prevent contamination by veneral diseases.

These medications can also be successfully used for the prevention of grippe and flu viral infections after contact with a sick person by using for washing of the mouth and throat and dropping in the nose or inhale 2-3 sprays nasally and throat twice a day of the solution “B” (Brilman's solution) with following application of cream or gel Pantosept and 0.2% or cream (or gel), Oropant, containing 0.2% Pantocidi (Pantosept) and 0.2% Oxolini, to mucous membranes in the nose. Solution Oxopant for inhalation can be used as medical and preventive means for grippe and flu.

What I claim as my invention is:

1. For preventive maintenance of the contamination viruses HIV/AIDS infection, grippe and flu. I suggest externally applying the solution “B” (Brilman's solution) including Pantocidi (Pantosept)—0.2%, Sodium Chloride—4%, Hydrocarbonate Sodium—5% and Sodium Tetraborate—0.5% on distilled water. Subsequently also use externally the proposed unguent (cream or gell) Pantosept 0.2%, or cream and solution “Oxopant” (containing 0.2% Pantocidi and 0.2% Oxolini).
